(19) United States
(12) Patent Application Publication
(10) Pub. No.: US 2009/0047220 A1
(43) Pub. Date: Feb. 19, 2009

(54) CONTRAST MEDIUM FOR ADMINISTRATION TO A PATIENT FOR MAGNETIC RESONANCE IMAGING

(75) Inventor: GA-LANE CHEN, Santa Clara, CA (US)

Correspondence Address: PCE INDUSTRY, INC.;ATT. CHENG-JU CHIANG, 458 E. LAMBERT ROAD, FULLERTON, CA, 92835, US

(73) Assignee: HON HAI PRECISION INDUSTRY CO., LTD., Tu-Cheng (TW)

(21) Appl. No.: 11/946,447

(22) Filed: Nov. 28, 2007

(30) Foreign Application Priority Data
Aug. 13, 2007 (CN) 200710201347.2

(51) Int. Cl. A61K 49/18

(52) U.S. Cl. 424/9.32; 977/744

(57) ABSTRACT

An exemplary contrast medium for administration to a patient for magnetic resonance imaging is shown. The contrast medium includes: a plurality of carbon nanospheres; and an iron containing nano-particle embedded in each of the carbon nanospheres.

### BACKGROUND

1. Technical Field

The present invention relates to magnetic resonance imaging technologies, and particularly to a contrast medium for administration to a patient for magnetic resonance imaging.

2. Description of Related Art

Magnetic resonance imaging (MRI) was first carried out in 1973, it has been widely used in providing detailed information useful for differentiating, diagnosing, or monitoring structures or conditions of various body tissues. Magnetic resonance imaging has been proposed for application in physics, chemistry, biology, medicine and a variety of other fields.

Magnetic resonance imaging equipments utilize a magnet, a radio frequency generator, a magnetic resonance signal receiver and a computer for processing and generating images of body tissues. When a body, for example, a body of a patient is moved adjacent to the magnet, nucleus, such as H nucleus contained in the tissues in the body of the patient, which were in ruleless spin and generated ruleless magnetic moments will realign under a magnetic field generated by the magnet. Under this state, when the radio frequency generator sends a radio frequency pulse to stimulate the H nucleus, the H nucleus will absorb energy thereof and transit to an active state, this is called magnetic resonance phenomenon. After that, when the radio frequency generator stops working, the radio frequency pulse will disappear, and the H nucleus will release the absorbed energy and return to an initial state from the active state. This return process is called relaxation process, and a time of the relaxation process is called relaxation time. The H nucleus will emit electromagnetic waves during the relaxation process, and the magnetic resonance signal receiver will detect the electromagnetic waves signals from the H nucleus. The computer can process such signals and transform it into images, therefore the magnetic resonances of the H nucleus in the tissues in the body of the patient can be observed.

With the ongoing development of the magnetic resonance imaging, a higher resolution of the magnetic resonance imaging for some micro or special tissues is required, such that some contrast mediums, such as gadolinium base compounds are studied. These contrast mediums are usually paramagnetic and can be attached on the tissues. This helps accelerating the relaxation process, shortening the relaxation time of the H nucleus thereby enhancing image contrasts between normal tissues and abnormal tissues such as a cancerous tissue.

However, gadolinium element is poisonous, and some gadolinium base compounds still do harm to some special tissues.

What is needed, therefore, is a contrast medium for administration to a patient for magnetic resonance imaging, which is less harmful to various tissues in the body of the patient.

### SUMMARY

In a present embodiment, an exemplary contrast medium for administration to a patient for magnetic resonance imaging is provided. The contrast medium includes: a plurality of carbon nanospheres; and an iron containing nano-particle embedded in each of the carbon nanospheres.

In another present embodiment, another exemplary contrast medium for administration to a patient for magnetic resonance imaging is provided. The contrast medium includes: a plurality of carbon nanotube bundles, the carbon nanotube bundles being constructed of a plurality of carbon nanotubes cross linked; and an iron containing nano-particle embedded in each of the carbon nanotube bundles.

Other advantages and novel features of the present invention will become more apparent from the following detailed description of preferred embodiment when taken in conjunction with the accompanying drawings.

### BRIEF DESCRIPTION OF THE DRAWINGS

Many aspects of a contrast medium can be better understood with reference to the following drawings. The components in the drawing are not necessarily drawn to scale, the emphasis instead being placed upon clearly illustrating the principles of the present contrast medium. Moreover, in the drawings, like reference numerals designate corresponding parts throughout the several views.

FIG. 1 is a schematic view of a tissue and a contrast medium for administration to a patient for magnetic resonance imaging according to a present embodiment of the present invention.

### DETAILED DESCRIPTION OF THE PREFERRED EMBODIMENTS

Embodiments of the present invention will now be described in detail below with reference to the drawings.

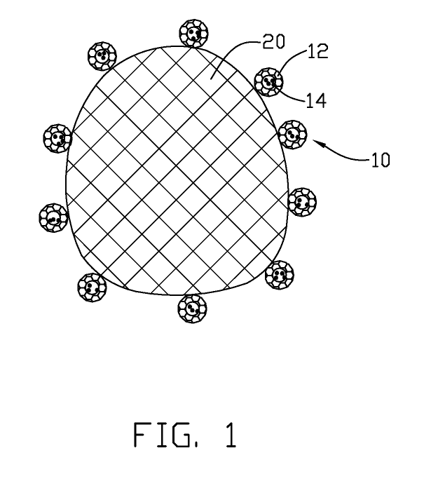

Referring to FIG. 1, a contrast medium 10 for administration to a patient for magnetic resonance imaging is shown. The contrast medium 10 includes a plurality of hollow carbon nanospheres 12 and a plurality of iron containing nano-particles 14 embedded in each of the hollow carbon nanospheres 12. The hollow carbon nanospheres 12 are attached on a tissue 20 in the body of the patient.

Each of the hollow carbon nanospheres is a polyhedral carbon cluster constructed with plurality of concentric graphitic sheets, and each of the concentric graphitic sheets has a closed spherical structure. An outer diameter of each of the hollow carbon nanospheres 12 is in a range from 100 nm to 2000 nm, preferably from 200 nm to 1000 nm. An inner diameter of each of the hollow carbon nanospheres 12 is in a range from 50 nm to 1200 nm, preferably from 50 nm to 850 nm.

The iron containing nano-particles 14 each can be selected from the group consisting of pure iron (Fe), iron oxides such as Fe₂O₃, FeO and other iron compounds. A particle size of each of the iron containing nano-particles 14 is in a range from 10 nm to 500 nm, preferably from 20 nm to 200 nm. The iron containing nano-particles 14 each are super paramagnetic due to the small particle size.

The hollow carbon nanospheres 12 and the iron containing nano-particles 14 can be formed synchronously, and at the same time, the iron containing nano-particles 14 are embedded in each of the hollow carbon nanospheres 12. The iron containing nano-particles 14 are packed and modified within the hollow carbon nano-spheres 12. The hollow carbon nanospheres 12 each have good water soluble property, such that the whole contrast medium 10 can be well dispersed in a water and then be injected or swallowed into the body of the patient. The hollow carbon nanospheres 12 and the iron containing nano-particles 14 do little harm to the tissue 20 in the body of the patient.

The hollow carbon nanospheres 12 each have a high specific surface area and low specific surface energy, such that they can be attached on the tissue 20 well. The whole contrast medium 10 can stay for a longer time in the body of the patient and not flow right away with the blood thereof, thereby helping to image the tissue 20.

Different areas of the tissue 20, for example, normal areas and abnormal areas such as cancerous areas may have different water containing capacity, i.e., have different H nucleus containing capacity. The abnormal area contain more H nucleus than the normal area, such that magnetic resonance signals of the abnormal area are higher than that of the normal area, thereby the abnormal area can be distinguished from the normal area. Due to super paramagnetic properties, the iron containing nano-particles 14 distribute at random on the tissue 20, and generate nonuniform magnetic fields in different areas thereof. The nonuniform magnetic fields accelerate the relaxation process of H nucleus of both of abnormal area and normal area, and shorten the relaxation time of the H nucleus thereof, thereby enhancing magnetic resonance signals of H nucleus of both of abnormal area and normal area, the abnormal area can be observed more clearly.

Alternatively, the hollow carbon nanospheres 12 each can be embedded with only one iron containing nano-particle 14 therein. The hollow carbon nanospheres 12 can be replaced by other hollow carbon nano-particles, for example, carbon nanotube bundles. Each of the carbon nanotube bundles is constructed with plurality of carbon nanotubes cross linked. The carbon nanotube bundles can be modified by water soluble polymers thereby facilitating being injected or swallowed into the body of the patient.

It is understood that the above-described embodiments are intended to illustrate rather than limit the invention. Variations may be made to the embodiments and methods without departing from the spirit of the invention. Accordingly, it is appropriate that the appended claims be construed broadly and in a manner consistent with the scope of the invention.

What is claimed is:

1. A contrast medium for administration to a patient for magnetic resonance imaging, the contrast medium comprising:
a plurality of carbon nanospheres; and
an iron containing nano-particle embedded in each of the carbon nanospheres.

2. The contrast medium as described in claim 1, wherein each of the carbon nanospheres is a polyhedral carbon cluster constructed of a plurality of concentric graphitic sheets, each of the concentric graphitic sheets having a closed spherical structure.

3. The contrast medium as described in claim 1, wherein each of the carbon nanospheres has an outer diameter in the range from 200 nm to 1000 nm.

4. The contrast medium as described in claim 1, wherein the iron containing nano-particle is comprised of pure iron or an oxide of iron.

5. The contrast medium as described in claim 4, wherein the oxide of iron is Fe₂O₃ or FeO.

6. The contrast medium as described in claim 1, wherein the iron containing nano-particle has a particle size in the range from 20 nm to 200 nm.

7. The contrast medium as described in claim 1, further comprising a carrier liquid, the carbon nanospheres with the iron containing nano-particle therein are dispersed in the carrier liquid.

8. A contrast medium for administration to a patient for magnetic resonance imaging, the contrast medium comprising:
a plurality of carbon nanotube bundles, the carbon nanotube bundles being constructed of a plurality of carbon nanotubes cross linked; and
an iron containing nano-particle embedded in each of the carbon nanotube bundles.

9. The contrast medium as described in claim 8, wherein each of the carbon nanotube bundles has an outer diameter in the range from 200 nm to 1000 nm.

10. The contrast medium as described in claim 8, wherein the iron containing nano-particle is comprised of pure iron or an of iron.

11. The contrast medium as described in claim 10, wherein the oxide of iron is Fe₂O₃ or FeO.

12. The contrast medium as described in claim 8, wherein the iron containing nano-particle has a particle size in the range from 20 nm to 200 nm.

13. The contrast medium as described in claim 8, further comprising a carrier liquid, the carbon nanotube bundles with the iron containing nano-particle therein are dispersed in the carrier liquid.
